(19) United States
(12) Patent Application Publication
(10) Pub. No.: US 2012/0220005 A1
(43) Pub. Date: Aug. 30, 2012

(54) METHOD FOR PRODUCING ETHANOL FROM LIGNOCELLULOSIC BIOMASS

(75) Inventors: Chikanori Kumagai, Kobe-shi (JP); Noriyuki Taniyama, Kobe-shi (JP); Yoshitoshi Nakamura, Tokushima-shi (JP)

(73) Assignees: THE UNIVERSITY OF TOKUSHIMA, TOKUSHIMA (JP); KAWASAKI JUKOGYO KABUSHIKI KAISHA, HYOGO (JP)

(21) Appl. No.: 13/393,518

(22) PCT Filed: Sep. 2, 2009
(86) PCT No.: PCT/JP2009/004318

§ 371 Date: May 15, 2012

(51) Int. Cl. C12P 7/06

(52) U.S. Cl. 435/161

(57) ABSTRACT

An object of the invention is to provide an inexpensive and efficient ethanol production method using lignocellulosic biomass as a raw material.

The method for producing ethanol from lignocellulosic biomass of the present invention includes
a lignocellulose decomposition step of subjecting lignocellulosic biomass to a steam explosion treatment or hydrolysis in a subcritical state followed by a flash treatment;
a lignin removal step of immersing a solid residue resulting from the decomposition step in ethanol to remove lignin; and
a C6 saccharification/simultaneous fermentation step of saccharifying the solid residue resulting from the lignin removal step with an enzyme and further fermenting the resulting product into ethanol with a C6 fermentation microorganism.

By combining the steam explosion treatment and the lignin removal process by ethanol immersion, higher rates are achieved for the saccharification and fermentation of the biomass.

### TECHNICAL FIELD

The present invention relates to a method for producing ethanol using hemicellulose or cellulose contained in lignocellulosic biomass such as wood-based biomass and soft biomass as a raw material by inexpensive and efficient saccharification, followed by ethanol fermentation using yeast.

### BACKGROUND ART

Lignocellulosic biomass such as a wood-based biomass is constituted with about 20% of hemicellulose, about 50% of cellulose, and about 30% of lignin. Hemice1lulose and cellulose can be decomposed into sugars by saccharification and further fermented by using a fermentation microorganism such as yeast to produce ethanol. The saccharification of hemicellulose results in C5 sugars and C6 sugars and the saccharification of cellulose results in C6 sugars.

As referred to herein, the term “C5 sugars” means pentose such as xylose and arabinose and oligosaccharides thereof, and the term “C6 sugars” means hexose such as glucose and galactose and oligosaccharides thereof.

Typical saccharification methods of lignocellulosic biomass include a concentrated sulfuric acid process and a dilute sulfuric acid process. However, the concentrated sulfuric acid process requires expensive equipments with high level of acid resistance since sulfuric acid of high concentration from 70% to 80% is used at a temperature of about 50° C. to about 100° C. although high saccharification efficiency can be achieved. It is also costly for recovery of sulfuric acid. The dilute sulfuric acid process is disadvantageous in very low saccharification rate (25% to 40%) for cellulose, although high saccharification rate (65% to 90%) is attainable for hemicellulose (NPL 2 and NPL 3).

When lignocellulosic biomass is hydrolysed with dilute sulfuric acid at a temperature between 150° C. and 180° C. for a few minutes, hemicellulose is first hydrolyzed to yield C5 sugars, i.e., pentose such as xylose and arabinose and their oligosaccharides and C6 sugars, i.e., hexose such as glucose, galactose and mannose and their oligosaccharides (first saccharification step).

The saccharification of hemicellulose results in a solid residue constituted with lignin and cellulose. When this residue is hydrolysed with dilute sulfuric acid at a temperature between 230° C. and 250° C. for 1 to 3 minutes, hexose such as glucose and its oligosaccharides can be obtained from the cellulose (second saccharification step).

In the first saccharification step, the saccharification rate of hemicellulose is not less than 80%, while in the second saccharification step the saccharification rate of cellulose is as low as about 30% to about 40%.

The two-step saccharification method as described above uses a carbohydrate material as a slurry form in each of the first and second saccharification steps, leading to a low ethanol concentration after ethanol fermentation. This causes an increase in energy consumption in an ethanol distillation step making it difficult to achieve high energy efficiency.

Due to a high treatment temperature in the second saccharification step, it is known that the solid residue of a hemicellulose component failed to be saccharified in the first saccharification step may be further decomposed into organic acids such as acetic acid and formic acid that are fermentation inhibitors, and levulinic acid, furfural, 5-HMF and the like may also be produced, whereby adverse effects may be exerted on alcoholic fermentation that subsequently follows.

To solve these technical problems in the art of saccharification by the concentrated sulfuric acid process or the dilute sulfuric acid process, the attempts to saccharify a cellulose component with an enzyme are being in progress as disclosed in PTLs 1 to 5. In these techniques, as a pretreatment prior to the enzymatic saccharification, lignocellulosic biomass is treated by acid hydrolysis, and then subjected to solid-liquid separation and further the solid residue is crushed, pulverized, etc. or treated with an alkali agent.

In addition, PTL 5 discloses a method for producing ethanol in which after hemicellulose is treated by acid hydrolysis, the saccharified solution is neutralized and fermented, while the residue is crushed, enzymatically hydrolysed and then fermented to produce ethanol.

### CITATION LIST

### Patent Literature

PTL 1: Japanese Laid-Open Patent Application Publication No. 2008-43328
PTL 2: Japanese Laid-Open Patent Application Publication No. 2008-161137
PTL 3: Japanese Laid-Open Patent Application Publication No. 2007-104983
PTL 4: Japanese Laid-Open Patent Application Publication No. 2007-151433
PTL 5: Japanese Laid-Open Patent Application Publication No. 2006-246711

### Nonpatent Literature

NPL 1: Carlo N. Hamelinck, Geertje van Hooijdonk, Andre P. C. Faaij, Prospects for ethanol from lignocellulosic biomass: techno-economic performance as development progresses, Science Technology Society, November 2003, P15 (ISBN 90-393-2583-4).
NPL 2: “Bioethanol Production Technique”: Japan Alcohol Association, Kogyo Chosakai Publishing Co., Ltd, issued in December, 2003.
NPL 3: “Characteristics and Energy Conversion and Utilization of Biomass”, NTN Publishing Co. Ltd., issued on Apr. 30, 2002.

### SUMMARY OF INVENTION

### Technical Problem

However, the conventional art, in which lignocellulosic biomass is hydrolysed with dilute sulfuric acid to produce sugars, has the following problems: (1) it results in low saccharification rate since the produced sugars are further decomposed; (2) it is difficult to achieve economic efficiency since the ethanol fermentation results in low ethanol concentration, causing high energy consumption during an ethanol distillation step; (3) the saccharification of hemicellulose and that of cellulose allow the lignin to be dissolved, which inhibits ethanol fermentation that subsequently follows; (4) simultaneous fermentation of C5- and C6-saccharified solution is difficult (only C6 saccharified solution is turned into ethanol).

In addition, as disclosed in NPL 1, enzymatic saccharification of lignocellulosic biomass such as soft biomass has an advantage that it does not cause decomposition of the produced sugars. However, its problem lies in that the economical production of ethanol is hardly attainable since the lignin covering cellulose cannot be sufficiently removed, preventing efficient enzymatic fermentation and therefore requiring a large amount of enzyme.

An object of the present invention is to provide an inexpensive and efficient method for producing ethanol using lignocellulosic biomass as a raw material.

### Solution to Problem

The present inventors have found that with respect to the solid residue resulting from a steam explosion treatment or a treatment in a subcritical state of the lignocellulosic biomass, the lignin covering cellulose can be removed through immersion in ethanol, and thus the present invention was accomplished.

Specifically, the present invention provides a method for manufacturing ethanol from lignocellulosic biomass, characterized by including:

a lignocellulose decomposition step, in which after the lignocellulosic biomass is subjected to a steam explosion treatment or hydrolyzed in a subcritical state, the resulting product is subjected to a flash treatment;

a lignin removal step of removing lignin by ethanol immersion of the solid residue resulting from the decomposition step; and

a C6 saccharification fermentation step of saccharifying the solid residue resulting from the lignin removal step with an enzyme and further fermenting the resulting product into ethanol.

In the present invention, lignocellulose biomass is subjected to a steam explosion treatment and saccharified, and solid-liquid separation is carried out to separate into a saccharified solution (liquid phase) and a solid residue. Then, C5 sugars are sufficiently recovered by washing the solid residue with water. The wash water containing C5 sugars and the saccharified solution (solution subjected to a steam explosion treatment or solution treated in a subcritical state) resulting from the solid-liquid separation are mixed together to prepare a C5 saccharified solution.

Cellulose that is not saccharified following the steam explosion treatment remains in the solid residue washed with water, and it is difficult to decompose highly efficiently into C6 sugars by saccharification of the cellulose as it is by adding an enzyme such as meicelase since the cellulose is covered with lignin. In contrast, the present invention improves the efficiency of enzymatic saccharification of cellulose by dissolving and removing lignin through immersing the solid residue in ethanol.

It is preferable to include a C5 fermentation step of carrying out the ethanol fermentation of the C5 saccharified solution using C5 yeast after the liquid phase resulting from the decomposition step was concentrated.

C5 sugars (for example, xylose, arabinose, or xylo-oligosaccharide) are contained in the saccharified solution (liquid phase) and in the wash water obtained by washing the solid residue; however, since the C5 sugar concentration is too low, a reverse osmosis membrane device is used to be increased the concentration to about 3% to about 6%. The concentration procedure increases the speed of the ethanol fermentation with a C5 fermentation microorganism.

It is preferred that after separating the fermentation microorganism (C5 fermentation microorganism) from the C5 fermented liquid resulting from the C5 fermentation step described above, the C5 fermented liquid be supplied to C6 saccharification/simultaneous fermentation step.

The maximum concentration of ethanol is about 3% for the C5 fermentation and 15% for the C6 fermentation. When ethanol is produced from biomass in an integrated plant, the final concentration of ethanol is preferably as high as possible. After separating a C5 fermentation microorganism by means of a centrifugal separator from the C5 fermented liquid having an ethanol concentration of about 3% obtained after completing the ethanol fermentation, the resulting C5 fermented liquid is combined with the C6 saccharified solution. Thus, as compared with the case in which C5 saccharified solution and C6 saccharified solution are fermented separately, higher ethanol concentration before an ethanol distillation step can be achieved, leading to reduction in energy consumption during the ethanol distillation step.

It is preferable to further include an immersion step of immersing the lignocellulosic biomass in ethanol or aqueous ammonia prior to the decomposition step.

Immersing the lignocellulosic biomass in ethanol or aqueous ammonia prior to the decomposition step enables the concentration of substances to be decreased which are harmful to ethanol fermentation process such as furfural, 5-hydroxymethylfurfural (5-HMF) and organic acid formed during the steam explosion treatment or hydrolysis in a subcritical state.

With respect to ethanol, anhydrous ethanol may be used and an aqueous solution having a concentration of not less than 20% may be used. Also, aqueous ammonia having a concentration of not greater than 20% is preferably used. In addition, immersion time period is preferably not shorter than 1 hour and not longer than 24 hours.

It is preferable to reuse the C5 fermentation microorganism separated from the C5 fermented liquid for the ethanol fermentation of the C5 saccharified solution. Similarly, it is preferable to separate the C6 fermentation microorganism from the C6 fermented liquid after the C6 saccharification/simultaneous fermentation step and reuse the microorganism for the ethanol fermentation of the C6 saccharified solution.

The ethanol production costs can be reduced by reusing the fermentation microorganism such as yeast.

The foregoing objects, other objects, characteristics and advantages of the present invention will be clear from detailed description of preferred embodiments given below with reference to attached drawings.

### Advantageous Effects of Invention

In the method for producing ethanol from lignocellulosic biomass of the present invention, a steam explosion treatment and ethanol immersion are combined to achieve an increase in the saccharification rate of cellulose by a C6 enzyme, which was conventionally insufficient. In addition, when a C5 fermented liquid is mixed with the solid residue resulting from the steam explosion treatment and the mixture is subjected to saccharification/simultaneous fermentation, the overall concentration of ethanol produced in the entire integrated plant can be increased up to 8%. Further, it is also possible to increase the ethanol yield by immersing the lignocellulosic biomass in aqueous ammonia or ethanol as a pretreatment prior to the steam explosion treatment for reducing the amount of inhibitors of ethanol fermentation produced during the steam explosion treatment.

### BRIEF DESCRIPTION OF DRAWINGS

FIG. 1 shows a process flow chart of Embodiment 1 of the present invention.

FIG. 2 shows a conceptual diagram illustrating a steam explosion device.

FIG. 3 shows a process flow chart of Embodiment 2 of the present invention.

### DESCRIPTION OF EMBODIMENTS

Hereinafter, embodiments of the present invention are explained with appropriate reference to the drawings. It should be noted that the present invention is not limited to the descriptions given below.

### Embodiment 1

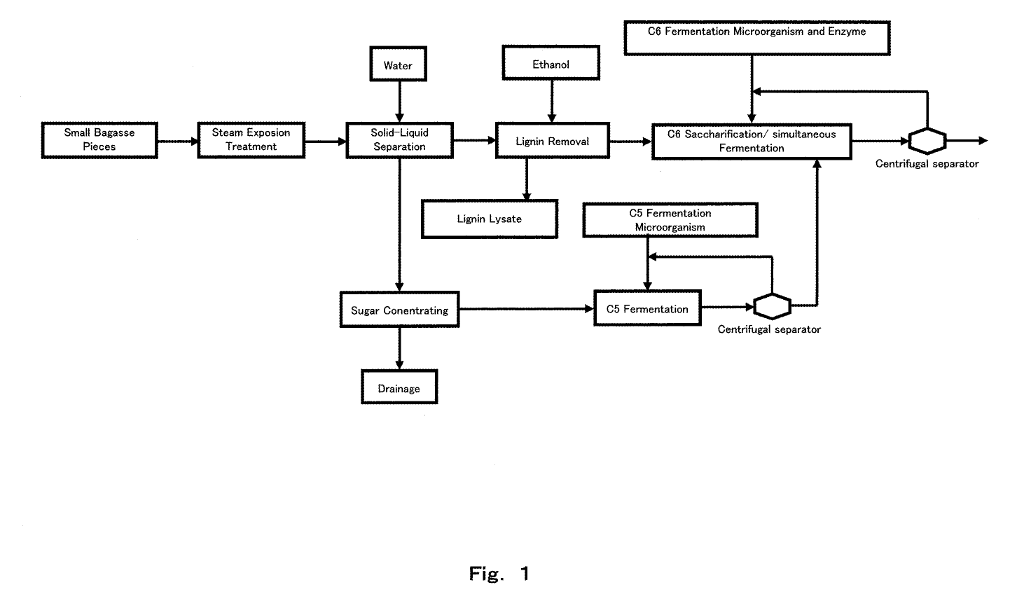

The process flow chart according to Embodiment 1 of the present invention is shown in FIG. 1.

(Decomposition Step)

First, lignocellulosic biomass (hereinafter, referred to as “biomass”) such as sugar cane bagasse is broken into small pieces to give a mean diameter of not greater than 30 to 50 mm (preferably, not greater than 10 mm) with a crushing machine or a grinding machine, etc. Hemicellulose component in the biomass is saccharified by subjecting these small pieces to a steam explosion treatment using steam in a steam explosion device (200 to 240° C., 1.5 to 4 MPa, 1 to 15 minutes; preferably, 225 to 230° C., 2.5 to 3 MPa, 1 to 5 minutes).

The biomass treated by the steam explosion device (steam-exploded material) includes C5 saccharified solution derived from hemicellulose, fermentation inhibitors such as decomposed sugar components and lignin lysate, and solid residues. The steam-exploded materials are separated into steam-exploded solution and a solid residue by solid-liquid separation using a filter press. The solid residue is further washed with water arbitrarily to recover sugars contained in the solid residue. The washing with water improves the recovery rate of the sugar.

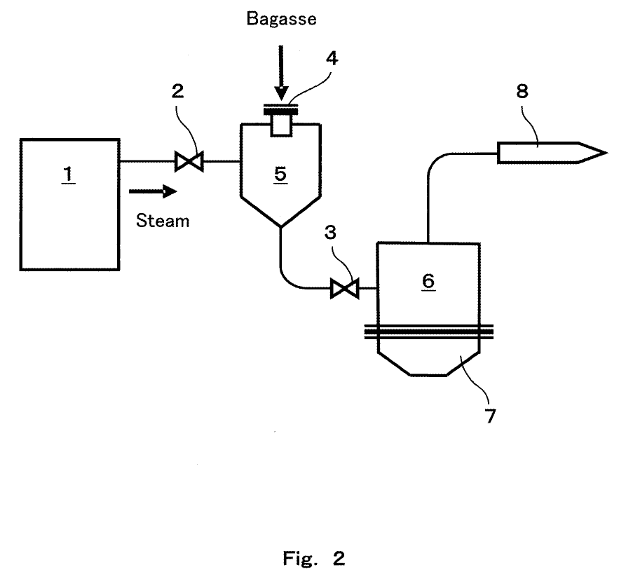

In this regard, the conceptual diagram of the steam explosion device is shown in FIG. 2. The heating temperature of a boiler is determined from the saturated steam curve such that a predetermined blasting pressure is attained, and operation of boiler 1 is started. Next, valve 2 and valve 3 are set to “close”. After adding a predetermined quantity of bagasse through an input port 4, the system is sealed up. Then, the valve 2 is set to “open”, and the steam from the boiler 1 is supplied to a reactor 5 and treated with heat.

After being treated with heat for a predetermined period of time, the valve 2 is set to “close” and immediately the valve 3 is set to “open” to carry out the steam explosion treatment. Upon the pressure release, solid materials (solid residue) and steam-exploded solution are transported into a separator 6, and separated from the steam. Then, the steam-exploded solution and solid materials are collected in a receiver 7. Subsequently, the receiver 7 is detached and the steam-exploded solution and solid materials are recovered.

(C5 Fermentation Step)

Next, the wash water containing C5 sugars is mixed with the steam-exploded solution to give a C5 saccharified solution. Mixing the wash water decreases the sugar concentration of the saccharified solution. In this regard, sugar concentrating is carried out on the C5 saccharified solution using a membrane such as a reverse osmotic membrane, capable of concentrating C5 sugars. As the sugar concentration increases, the speed of the fermentation of the C5 saccharified solution increases. In addition, due to the reduction in quantity of the saccharified solution, a downsizing of the fermentor for the C5 saccharified solution can be also realized.

After the sugar concentrating, the C5 saccharified solution is transferred to the fermentor used for the C5 saccharified solution, in which ethanol fermentation is carried out with a fermentation microorganism for the C5 saccharified solution (a C5 fermentation microorganism: e.g., Pichia stipitis) at 27 to 35° C. for 48 to 72 hours.

The C5 saccharified solution is turned into a C5 fermented liquid by ethanol fermentation, capable of allowing for the ethanol concentration of up to about 3%. After the C5 fermentation microorganism is separated by a centrifugal separator, it is possible to take the C5 fermented liquid out of the plant as it is. However, in the present invention, it is preferable to mix the liquid with the solid residue resulting from the steam explosion treatment and simultaneously subject to saccharification and ethanol fermentation by means of the enzyme and the fermentation microorganism for the C6 fermented liquid (C6 fermentation microorganism), as described later.

Also, in the case in which the C5 saccharified solution contains xylo-oligosaccharide, externally adding an enzyme such as xylanase decomposes the xylo-oligosaccharide into monosaccharides, thereby improving the ethanol fermentation rate of the C5 saccharified solution.

(Lignin Removal Step)

After the solid residue obtained by the steam explosion treatment is washed with water to remove sugars and lignin lysate, and the resulting residue is immersed in aqueous ethanol having an ethanol concentration of not less than 30% and preferably not less than 50%, at room temperature for the period of time not shorter than 0.5 hours and not longer than 48 hours and preferably not shorter than 1 hour and not longer than 24 hours. Accordingly, the lignin covering the cellulose is dissolved and subsequently removed. After the ethanol immersion, the solid residue may be subjected to a mechanical ethanol elimination treatment or an ethanol elimination treatment by heating or heating under reduced pressure, etc. Note that when aqueous ethanol having a high concentration is used, the ethanol elimination treatment may be carried out after washing with water once.

(C6 Fermentation Step)

The solid residue resulting from the ethanol elimination treatment is transferred to the vessel used for the C6 saccharification/simultaneous fermentation. In this step, it is preferable that the aforementioned C5 fermented liquid be added to produce a slurry having a predetermined concentration (10 to 20%) of the solid content. A predetermined quantity (the ratio of carbohydrate material to the enzyme: between 5 to 1 and 2000 to 1, and preferably between 10 to 1 and 1000 to 1; cell mass content of fermentation microorganism: not less than 0.1 w/v % and not greater than 3 w/v % and preferably not less than 0.3 w/v % and not greater than 1.5 w/v % with respect to the quantity of fermentation liquid) of the enzyme or the C6 fermentation microorganism (for example, Saccharomyces cerevisiae) are charged and the ethanol fermentation is permitted at 27 to 35° C. for 24 to 48 hours.

In other words, according to the C6 saccharification/simultaneous fermentation step of the present invention, it is preferable to produce a slurry using not water but the C5 fermented liquid instead. Since the C6 fermentation microorganism is superior in resistance to organic acids and ethanol compared with the C5 fermentation microorganism, even if a slurry of the solid residue is produced using the C5 fermented liquid, the C6 saccharification/simultaneous fermentation is possible.

In the solid residue resulting from the steam explosion treatment, microfibrils within the cellulose are loosened because of a great deal of power mechanically applied by the instantaneous release of pressure. Furthermore, ethanol immersion removes lignin to the most. As a result, enzymes such as cellulase can easily enter into the microfibrils and facilitate the saccharification reaction as compared with conventional techniques, whereby a higher ethanol yield can be achieved.

In the C6 saccharification/simultaneous fermentation vessel, since both cellulase and the C6 fermentation microorganism are present admixed, even in the case in which the saccharification reaction of cellulose progresses, the fermentation microorganism maintains a very low concentration of C6 sugars such as glucose, thereby achieving stable ethanol fermentation.

<1. Improvement in Saccharification Rate by Ethanol Immersion>

Investigated were the variations in the enzymatic saccharification rate of cellulose in the C6 saccharification/simultaneous fermentation step, owing to the combination of the steam explosion treatment and the lignin removal step by ethanol immersion, using 100 g of small sugar cane bagasse pieces each having about 30-mm side in size as a raw material. In the ethanol immersion process, after washing with water the solid residue resulting from the steam explosion treatment, the resultant was immersed in anhydrous ethanol at room temperature for 1 hour. Note that the enzymatic saccharification rate of cellulose was calculated according to the following formula:

[((Quantity of Produced C6 Sugars×0.9)/Cellulose Quantity)×100]. The results are shown in Table 1.

TABLE 1

| Steam | | Enzymatic |
|---|---|---|
| explosion | Post Steam explosion | Saccharification |
| treatment | treatment | Rate of Cellulose |
| None | None | 8.5% |
| None | Water Wash/Ethanol | 20.8% |
| | Immersion | |
| 25 atm × 5 min | None | 87.9% |
| 30 atm × 5 min | None | 79.5% |
| 35 atm × 5 min | None | 73.6% |
| 25 atm × 5 min | Water Wash/Ethanol | 87.9% |
| | Immersion | |
| 30 atm × 5 min | Water Wash/Ethanol | 100.0% |
| | Immersion | |
| 35 atm × 5 min | Water Wash/Ethanol | 90.5% |
| | Immersion | |

The enzymatic saccharification rate of cellulose was less than 10% with carrying out neither a steam explosion treatment nor ethanol immersion, and was about 20% with carrying out only ethanol immersion. Also when the steam explosion treatment was performed at 25 to 35 atm for 5 minutes, the enzymatic saccharification rate of cellulose was 73.6 to 87.9% without carrying out the ethanol immersion, but with the ethanol immersion carried out after the same steam explosion treatment, at least 20% increase in the enzymatic saccharification rate of cellulose was achieved in the case of 30 atm and 35 atm. In particular, under the conditions of 30 atm and 5 minutes, the cellulose was completely decomposed, i.e., the enzymatic saccharification rate of cellulose of 100% was accomplished.

The removal of lignin from the solid residue resulting from a steam explosion treatment facilitates enzymatic saccharification and leads to a higher C6 saccharification and higher fermentation rate. As a result, the ethanol production per unit weight of the raw material, namely biomass, can be as large as 200 L per ton of biomass, approximately the double of the case of acid hydrolysis method using a dilute sulfuric acid.

<2. Ethanol Concentration and Rate of Lignin Removal>

Investigated were the variations in the lignin removal rate caused by changes in the ethanol concentration and time of ethanol immersion under the conditions of 35 atm and 5 minutes described in the above section 1, using 100 g of small sugar cane bagasse pieces. The lignin removal rate was estimated based on changes in the weight. The results are shown in Table 2. Note that the unit of all the values in Table 2 is represented by percentage.

TABLE 2

| | | 50% | 30% |
|---|---|---|---|
| | | Aqueous | Aqueous |
| | Ethanol | Ethanol | Ethanol |
| 1 hour | 63.6 | 46.8 | 15.2 |
| Immersion | | | |
| 24 hour | 82.4 | 71.2 | 24.0 |
| Immersion | | | |
| 48 hour | 67.6 | 44.0 | 27.6 |
| Immersion | | | |

In the case of 30% aqueous ethanol, even with a longer immersion time period, the lignin removal rate was less than 30%. In the case of 50% aqueous ethanol and anhydrous ethanol, the maximum lignin removal rate was achieved with the immersion time of 24 hour. Repeating similar experiments led to the finding that it is preferable to adjust the ethanol concentration to not less than 50%, and the immersion time not shorter than 1 hour and not longer than 24 hours.

<3. Immersion in Aqueous Ethanol or Aqueous Ammonia Prior to Steam Explosion treatment, and Concentration of Harmful Substances>

Small pieces of sugar cane bagasse the same as those in section 1 above in an amount of 100 g of were immersed in anhydrous ethanol, 20% aqueous ethanol, or 20% aqueous ammonia at room temperature for 24 hours as a pretreatment prior to the steam explosion treatment. Subsequently, the steam explosion treatment was carried out similarly to section 1 above, and the sugar concentration (w/v %) and the concentration (mg/L) of the total of 3 types of decomposed sugar components: furfural, 5-HMF, and organic acid were determined. The results are shown in Table 3. Note that the sugar concentration and the concentrations of the 3 types of the decomposed sugar components were determined using high performance liquid chromatography.

TABLE 3

| | | Composition of Steam-exploded | |
|---|---|---|---|
| | Pretreatment | Liquid | |
| Steam Explosion | Prior to Steam | Sugar | Concentration of |
| Treatment | Explosion | Concentration | Decomposed Sugar |
| Conditions | treatment | (w/v %) | Components (mg/L) |
| 20 atm × 2 min | None | 0.48 | 343 |
| | Immersion in | 0.61 | 359 |
| | 20% Aqueous | | |
| | Ethanol | | |
| | Immersion in | 0.21 | 439 |
| | 20% Ammonia | | |
| | Solution | | |
| 35 atm × 3 min | None | 0.4 | 13,400 |
| | Immersion in | 0.9 | 3,304 |
| | Anhydrous | | |
| | Ethanol | | |
| | Immersion in | 0.23 | 666 |
| | 20% Ammonia | | |
| | Solution | | |

When the steam explosion treatment was carried out at 20 atm for 2 minutes, no significant changes in the concentration of the decomposed sugar components was found. However, when the steam explosion treatment was carried out at 35 atm for 5 minutes, the concentration of the decomposed sugar components was reduced to about one-fourth in the case of anhydrous ethanol immersion, and to about one-twentieth in the case of 20% aqueous ammonia immersion. In addition, in the case of anhydrous ethanol immersion, the sugar concentration was two times or more as compared with the case of no pretreatment involved.

<4. Addition of Ethanol in Preparation of Slurry for C6 Saccharification/Simultaneous Fermentation>

After 100 g of the small sugar cane bagasse pieces the same as those in the section 1 was subjected to the steam explosion treatment and the solid residue was washed with water, the resulting matter was immersed in anhydrous ethanol at room temperature for 3 hours. Ethanol was removed by washing with water and the slurry for C6 fermentation was prepared using water or 2% aqueous ethanol. Cellulase and a C6 fermentation microorganism (Saccharomyces cerevisiae) were added, and saccharification and ethanol fermentation were performed under the conditions of: the concentration of solid content of 10%; the ratio of the solid content to the enzyme of 10 to 1; the saccharification/fermentation temperature of 37° C.; and the saccharification/fermentation time of 48 hours. The weight of ethanol produced per unit weight of the raw material cellulose (ethanol production material unit: kg/kg) was determined according to the formula of:

[Wight of Ethanol Produced from Sugar Cane Bagasse Subjected to Steam Explosion Treatment/Weight of Cellulose in Sugar Cane Bagasse Subjected to Steam Explosion Treatment]. The results are shown in Table 4.

TABLE 4

| | C6 | |
|---|---|---|
| | Saccharification/ | Ethanol |
| | Simultaneous | Production |
| Steam Explosion | Fermentation | Material Unit |
| treatment | Slurry | (kg/kg) |
| 25 atm × 5 min | Prepared with | 0.301 |
| 35 atm × 5 min | Water | 0.282 |
| 25 atm × 2 min | Prepared with 2% | 0.318 |
| 35 atm × 1 min | Aqueous Ethanol | 0.389 |

The C6 fermentation slurry was prepared with 2% aqueous ethanol under the supposition of using the C5 fermented liquid obtained after removing the C5 fermentation microorganism; however it was found that even in the case in which ethanol was initially present in the slurry prepared for the C6 saccharification/simultaneous fermentation, the enzymatic saccharification/simultaneous fermentation was not inhibited.

### Embodiment 2

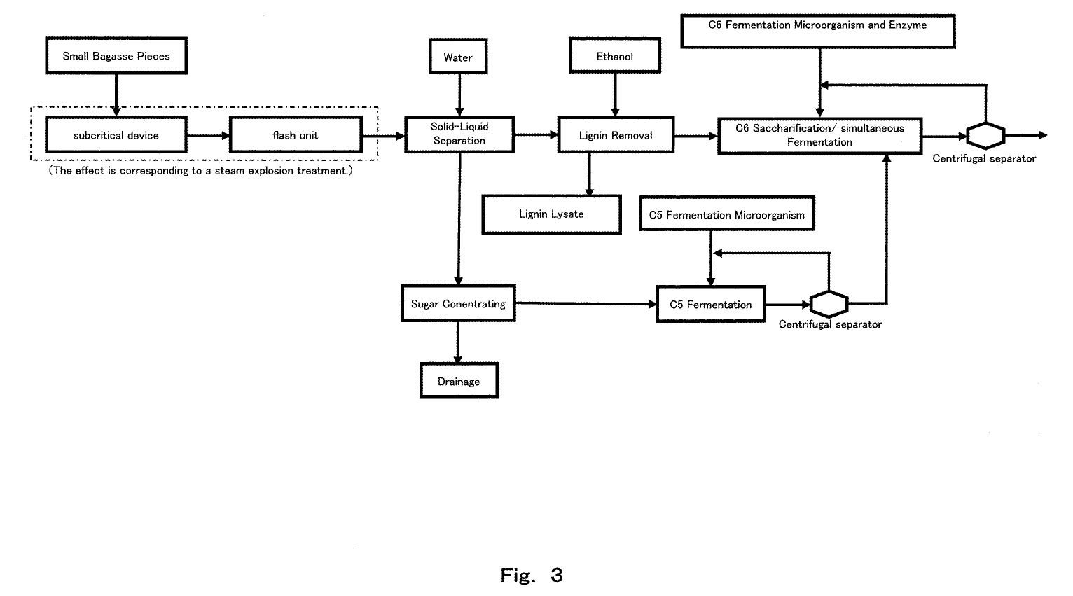

While biomass such as sugar cane bagasse small pieces is subjected to a steam explosion treatment with a steam explosion device in Embodiment 1, the steam explosion device can be replaced by a subcritical (water) device. For example, as shown in FIG. 3, in place of subjecting the bagasse small pieces to the steam explosion treatment, applying a sudden decrease in pressure by a flash unit after the small bagasse pieces were treated with hydrolysis at a subcritical state can achieve similar effects.

It is preferable to use the subcritical (water) device at a subcritical water temperature of 160 to 240° C. with the treatment time period of 1 to 90 minutes. The subcritical solvent is not limited to water, and an organic acid such as acetic acid (for example, at concentration of not greater than 0.1 M) or ethanol mixed solution may be used.

It should be noted that even in the case of using water as the subcritical solvent, performing ethanol immersion or aqueous ammonia immersion as a pretreatment prior to the subcritical treatment inhibits decomposition of the sugar, restraining the formation of organic acids such as furfural.

### Embodiment 3

In Embodiment 1, the biomass as a carbohydrate material was broken into small pieces to give a mean diameter of not greater than 30 to 50 mm (preferably, not greater than 10 mm) with a crushing machine or a grinding machine prior to the saccharification treatment. However, in the case in which the biomass is subjected to a steam explosion treatment under conditions of 25 to 35 atm and 5 minutes or longer, the biomass is ground into a fine powder of not greater than 100 μm; therefore, it is not necessary to give the mean diameter of not greater than 30 to 50 mm as long as the carbohydrate material can be ground into a size easily being handled in the later stage.

### Embodiment 4

It is also possible to use a fermentation microorganism displaying an enzyme on the surface thereof in place of the C5 fermentation microorganism and the C6 fermentation microorganism. Specifically, the use of a fermentation microorganism displaying xylanase or cellulase on the surface thereof, as disclosed in Japanese Laid-Open Patent Application Publication No. 2008-193935, enables the fermentation microorganism displaying the same enzyme on the surface to be employed in the C5 fermentation and C6 saccharification/simultaneous fermentation. In this case, only one fermentation microorganism vessel is required, leading to reduction in the equipment costs.

From the foregoing explanations, many improvements on and other embodiments of the present invention are apparent to a person skilled in the art. Therefore, the explanations above should be construed as illustrative examples provided for the purpose of explaining a person skilled in the art the best mode for carrying out the present invention. It is possible to substantially alter the details of the structure and/or functions without deviating from the spirit of the present invention.

### INDUSTRIAL APPLICABILITY

The method for producing ethanol from lignocellulosic biomass of the present invention is useful especially in the field of bioethanol production using a chemical plant.

### REFERENCE SIGNS LIST

1: Boiler

2, 3: Valve

4: Input port

5: Reactor

6: Separator

7: Receiver

8: Silencer

1. A method for producing ethanol from lignocellulosic biomass, the method comprising:
a lignocellulose decomposition step of subjecting lignocellulosic biomass to a steam explosion treatment or hydrolysis in a subcritical state;
a lignin removal step of removing lignin by ethanol immersion of the solid residue resulting from the decomposition step;
a C6 saccharification/simultaneous fermentation step of saccharifying the solid residue resulting from the lignin removal step with an enzyme and further fermenting the resulting product into ethanol with a C6 fermentation microorganism.

2. The method according to claim 1, further comprising a C5 fermentation step of carrying out ethanol fermentation of C5 sugars with a C5 fermentation microorganism after the liquid phase resulting from the decomposition step was concentrated.

3. The method according to claim 2, wherein after separating the fermentation microorganism from a C5 fermented liquid resulting from the C5 fermentation step, the C5 fermented liquid is supplied to the C6 saccharification/simultaneous fermentation step.

4. The method according to claim 3, wherein the C5 fermentation microorganism separated from the C5 fermented liquid is reused.

5. The method according to claim 1, further comprising an immersion step of immersing the lignocellulosic biomass in aqueous ammonia or ethanol prior the decomposition step.

6. The method according to claim 1, wherein the C6 fermentation microorganism is separated from the C6 fermented liquid resulting from the C6 saccharification/simultaneous fermentation step and reused.

7. The method according to claim 2, wherein in place of the C5 fermentation microorganism and the C6 fermentation microorganism, a fermentation microorganism displaying C5 enzyme and C6 enzyme on the surface thereof and capable of utilizing glucose and xylose to give ethanol.
